(19) United States
(12) Patent Application Publication
(10) Pub. No.: US 2015/0056146 A1
(43) Pub. Date: Feb. 26, 2015

(54) COMPOSITION USED FOR CONTRAST IMAGING

(71) Applicant: CANON KABUSHIKI KAISHA, Tokyo (JP)

(72) Inventor: Kouichi Kato, Kyoto-shi (JP)

(21) Appl. No.: 14/461,253

(22) Filed: Aug. 15, 2014

(30) Foreign Application Priority Data
Aug. 21, 2013 (JP) 2013-171646

(51) Int. Cl. A61K 49/22; A61K 49/00

(52) U.S. Cl. CPC A61K 49/22; A61K 49/0034; A61K 49/225
USPC 424/9.44; 424/9.6

(57) ABSTRACT

There is provided a composition used for contrast imaging, the composition containing an aqueous solution containing a J-aggregate of indocyanine green and at least one storage stabilizer selected from the group consisting of alkali metal ions, alkaline earth metal ions, and an ammonium ion.

### BACKGROUND OF THE INVENTION

1. Field of the Invention

The present invention relates to a composition used for contrast imaging, the composition containing a J-aggregate of indocyanine green.

2. Description of the Related Art

Indocyanine green (hereinafter also referred to as ICG) is a fluorescent dye which has the maximum absorption at a near-infrared wavelength.

The term “ICG” herein refers to a compound having a cyanine skeleton and a structure represented by Formula (1).

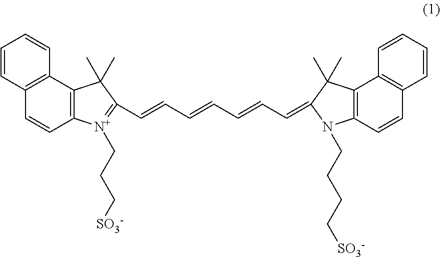

ICG is used as a medicament for internal diagnoses such as a liver function test, a circulatory function test, and identification of a sentinel lymph node in breast cancer. ICG is a medicament which can be safely administered to the human body for such diagnostic purposes and expected to be applied to, for example, a photoacoustic contrast agent.

A photoacoustic contrast agent is a contrast agent used for photoacoustic imaging. In photoacoustic imaging, light is emitted to an analytical area, light-absorbing molecules present in the area absorb the light and then emit heat, the heat causes volume expansion with the result that an acoustic wave is generated, and the intensity of the acoustic wave and the position at which the acoustic wave has been generated are detected to form an image of the analytical object. It is effective to use a photoacoustic contrast agent made of a material having a good optical absorption property to enhance the intensity of an acoustic wave emitted from an analytical area in photoacoustic imaging.

It is known that ICG forms a J-aggregate (hereinafter also referred to as J-ICG) under specific conditions (Chemical Physics; Volume 220; 1997; pp 385-392). The J-aggregate of ICG is one of ICG multimers; as compared to an ICG monomer (hereinafter simply referred to as ICG), the J-aggregate of ICG has the maximum absorbance shifted to the longer wavelength side, and the maximum absorbance is at a wavelength ranging from 880 nm to 910 nm. A J-aggregate of ICG and an ICG monomer are hereinafter referred to as J-ICG and ICG, respectively.

It is known that J-ICG has a sharp photoabsorption band; as compared to a monomer at the same concentration, the absorbance of the J-ICG at the maximum absorption wavelength is several times that of ICG. Thus, J-ICG is a material having a good optical absorption property.

Accordingly, such J-ICG is a good potential material of a photoacoustic contrast agent.

### SUMMARY OF THE INVENTION

The inventor has found the following: in the case where J-ICG is stored in water, the absorbance of this aqueous solution for light having a wavelength ranging from 880 nm to 910 nm decreases. It is believed that dissociation of the J-ICG into ICG monomers causes the decrease in the absorbance of the aqueous solution for light having a wavelength ranging from 880 nm to 910 nm.

In particular, in order to produce J-ICG used for a photoacoustic contrast agent, the storage stability of J-ICG in an aqueous solution needs to be enhanced.

An aspect of the present invention provides a composition used for contrast imaging, the composition containing an aqueous solution containing a J-aggregate of indocyanine green and at least one storage stabilizer selected from the group consisting of alkali metal ions, alkaline earth metal ions, and an ammonium ion.

Further features of the present invention will become apparent from the following description of exemplary embodiments with reference to the attached drawings.

### BRIEF DESCRIPTION OF THE DRAWINGS

FIG. 1 is a graph illustrating results of evaluation of the storage stability of J-ICG in reagents at 4° C.

FIG. 2 is a graph illustrating results of evaluation of the storage stability of J-ICG in reagents at 25° C.

FIG. 3 is a graph illustrating results of evaluation of the storage stability of J-ICG in reagents at 37° C.

FIG. 4 is a graph illustrating the relationship between the concentration of sodium chloride and the storage stability of J-ICG in an aqueous sodium chloride solution.

FIG. 5 is a graph illustrating results of evaluation of the storage stability of J-ICG in aqueous solutions of 10-mM alkali metal salts or in another liquid.

FIG. 6 is a graph illustrating results of evaluation of the storage stability of J-ICG in aqueous solutions of 1-mM alkali metal salts or in another liquid.

FIG. 7 is a graph illustrating results of evaluation of the storage stability of J-ICG in aqueous solutions of 0.1-mM alkali metal salts or in another liquid.

FIG. 8 is a graph illustrating the relationship between the concentrations of alkaline earth metal salts and results of evaluation of the storage stability of J-ICG in aqueous alkaline earth metal salt solutions.

### DESCRIPTION OF THE EMBODIMENTS

Embodiments of the present invention will now be described.

### Composition Used for Contrast Imaging

In a composition used for contrast imaging according to a first embodiment, an aqueous solution containing a J-aggregate of indocyanine green further contains a storage stabilizer. The inventor has studied a storage stabilizer that can enhance the underwater storage stability of J-ICG in an aqueous solution and found that alkali metal ions, alkaline earth metal ions, and an ammonium ion have an effect of an enhancement in the storage stability.

In this case, water is in the form of a specific structure (structured water) owing to intermolecular interaction; however, some water molecules form free water which can hydrate a solute. It is believed that J-ICG is hydrated by free water with the result the multimer is dissociated into monomers. ICG has a hydrophilic sulfate group (negative charge), and it is presumed that the sulfate group is hydrated with the result that the J-aggregate is dissociated in water.

The storage stabilizer suppresses the dissociation of J-ICG, and it is considered that the following two mechanisms bring such suppression.

First, alkali metal ions, alkaline earth metal ions, or ammonium ions attract free water to fix the free water to the vicinity of the ions. The amount of the free water around ICG is therefore reduced, and thus free water necessary for dissociation of J-ICG runs short, which results in a reduction in the dissociation of the J-ICG.

Secondly, the sulfate group contained in ICG is bonded to an alkali metal ion, an alkaline earth metal ion, or an ammonium ion with the result that hydration of the sulfate group is suppressed; thus, the dissociation of J-ICG is reduced.

Owing to these mechanisms, the composition used for contrast imaging according to the first embodiment has a high storage stability.

In the first embodiment, the storage stabilizer may be at least one selected from the group consisting of alkali metal ions, alkaline earth metal ions, and an ammonium ion.

Since alkaline earth metal ions are divalent, the amount of the positive charge thereof is twice as large as the amount of the positive charge of alkali metal ions and ammonium ion which are each monovalent; hence, the force for fixing free water is larger in alkaline earth metal ions than in alkali metal ions and an ammonium ion. Accordingly, the alkaline earth metal ions are more suitably employed as the storage stabilizer.

### J-ICG

In the first embodiment, any J-ICG which has the maximum absorbance at a wavelength ranging from 880 nm to 910 nm can be employed. In the case where J-ICG is in the form of a particle, the particle size of the J-ICG is normally in the range of 1 nm to 10 μm, and the J-ICG having such a particle size can be used for tumor imaging and lymph node imaging. In the first embodiment, the particle size of J-ICG is preferably in the range of 10 nm to 1 μm; in the case where the composition is used for tumor imaging, the particle size is more preferably not more than 100 nm. This is because particles having a particle size of not more than 100 nm are likely to accumulate at a tumor.

The composition used for contrast imaging is in the form of an injection on its use; in the case of a suspension injection which can be subcutaneously administered, the particle size is defined to be not more than 150 μm; in the case of an emulsion injection which can be intravascularly administered, the particle size is defined to be not more than 7 μm. Hence, the particle size of J-ICG which satisfies the above-mentioned range is applicable.

An example of production of J-ICG will now be described. An aqueous ICG solution at a concentration of 1.5 to 8 mM is heated at 70° C. for 12 hours in total and then left to stand at room temperature for 6 days or more to prepare the J-aggregate of ICG.

### Storage Stabilizer

The storage stabilizer used in the first embodiment may be at least one selected from the group consisting of alkali metal ions, alkaline earth metal ions, and an ammonium ion; and two or more of these can be used in combination.

Examples of the alkali metal ions include a lithium ion, a sodium ion, a potassium ion, a rubidium ion, and a cesium ion.

Examples of the alkaline earth metal ions include a beryllium ion, a magnesium ion, a calcium ion, a strontium ion, and a barium ion.

In view of being used for medicaments, at least one selected from the group consisting of a sodium ion, a potassium ion, a magnesium ion, a calcium ion, and an ammonium ion can be particularly used.

The storage stabilizer used in the first embodiment can be a salt of an alkali metal ion, an alkaline earth metal ion, or an ammonium ion within a pharmacological tolerance.

The molar concentration of the storage stabilizer used in the first embodiment is preferably at least 1 time, more preferably at least 1.25 times, and especially preferably at least 100 times the molar concentration of ICG.

In the case where the storage stabilizer used in the first embodiment is an alkaline earth metal ion, the molar concentration thereof can be at least 1 time that of ICG; in the case where the storage stabilizer is an alkali metal ion or an ammonium ion, the molar concentration thereof can be at least 100 times that of ICG.

The upper limit of the above-mentioned concentration is not particularly limited; however, the concentration needs to be at a level which enables the storage stabilizer to be dissolved in water.

The composition used for contrast imaging according to the first embodiment may further contain an additive other than the storage stabilizer.

Examples of usable additives include a pH adjuster such as a buffer solution and an isotonizing agent.

### Composition Used for Photoacoustic Imaging

A composition used for photoacoustic imaging according to a second embodiment contains the composition used for contrast imaging according to the first embodiment and a dispersion medium. The dispersion medium is a liquid material used for dispersing the compound employed in the second embodiment; examples thereof include distilled water for injection, phosphate buffered saline, and an aqueous glucose solution. An additive which can enhance the effect of a photoacoustic imaging, such as a material having a large coefficient of thermal expansion or a material having a small specific heat capacity, may be appropriately used.

In the composition used for photoacoustic imaging according to the second embodiment, the above-mentioned composition used for contrast imaging according to the first embodiment may be dispersed in the dispersion medium in advance. The above-mentioned composition used for contrast imaging according to the first embodiment and the dispersion medium may be in the form of a kit, and the composition used for contrast imaging may be dispersed in the dispersion medium before administration to a living body.

The composition used for photoacoustic imaging according to the second embodiment can be applied to optical imaging.

The term “optical imaging” herein refers to imaging (formation of image) by irradiation with light. In particular, the composition used for photoacoustic imaging according to the second embodiment is irradiated with light, thereby generating an acoustic wave or fluorescence. Detecting the generated acoustic wave enables photoacoustic imaging, and detecting the generated fluorescence enables fluorescent imaging. The photoacoustic imaging is an idea including photoacoustic tomography (planigraphy).

### EXAMPLES

Examples of the composition used for contrast imaging according to the present invention will now be described.

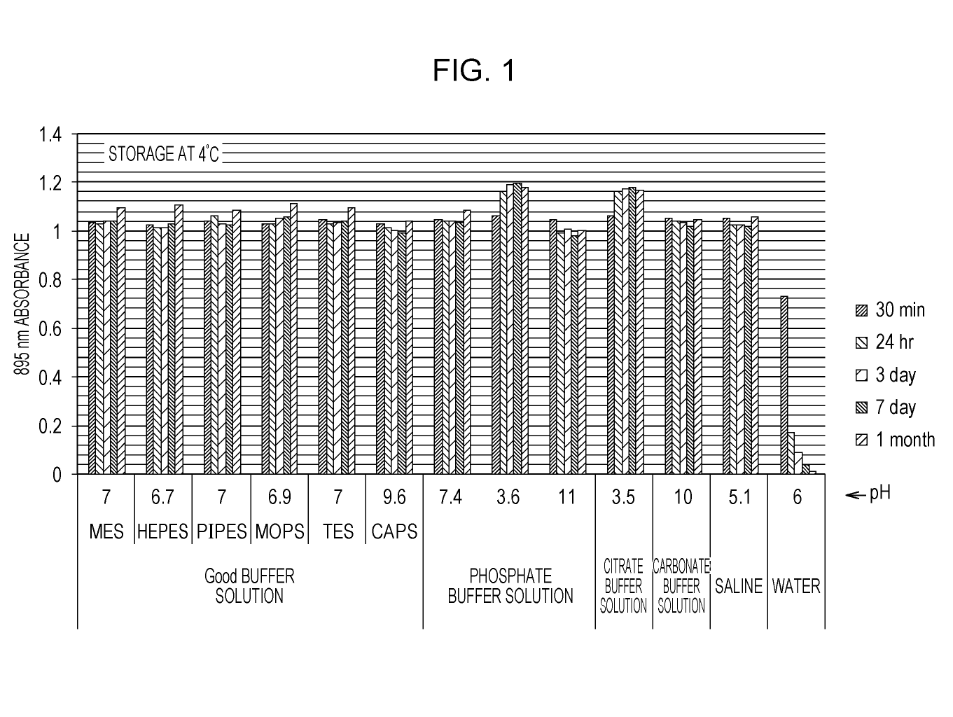

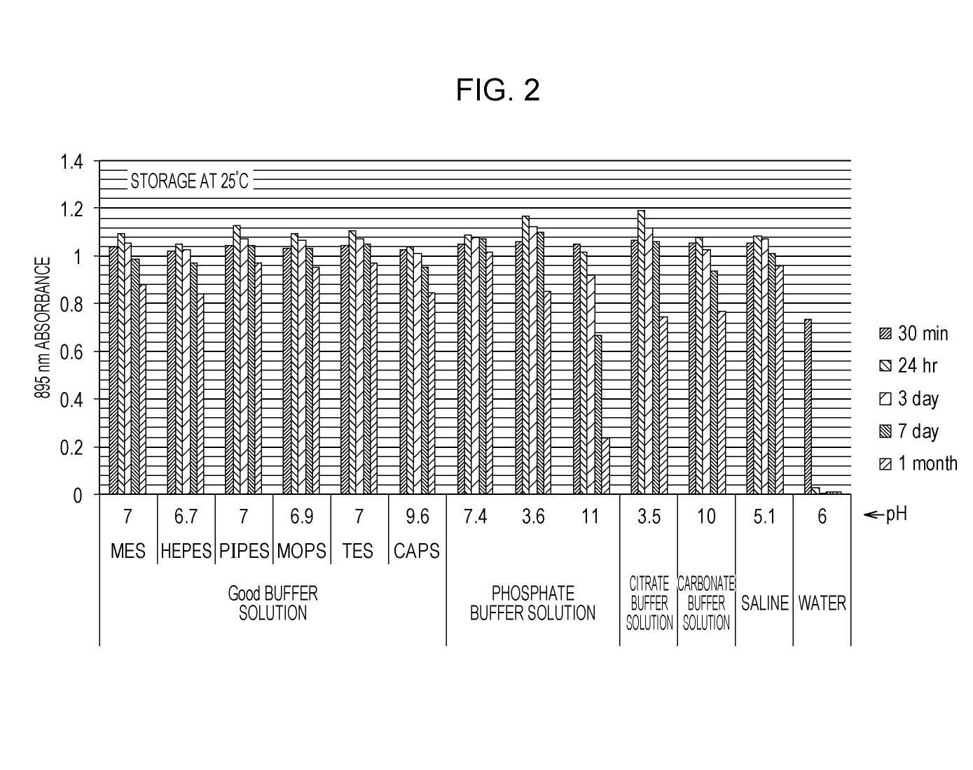

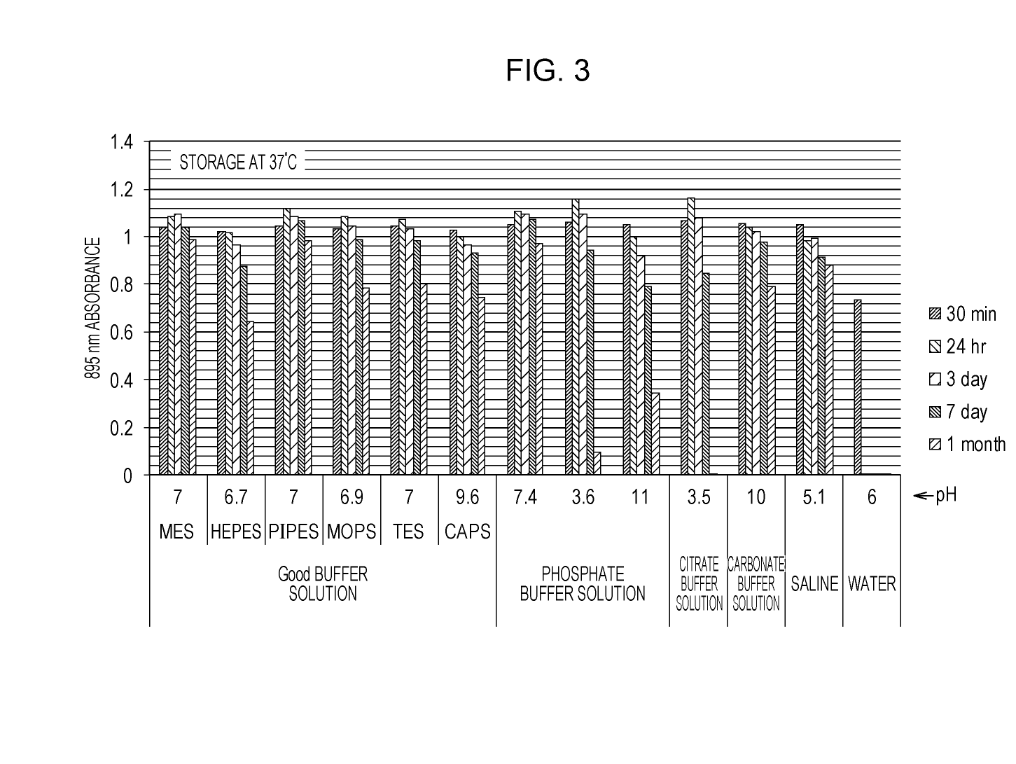

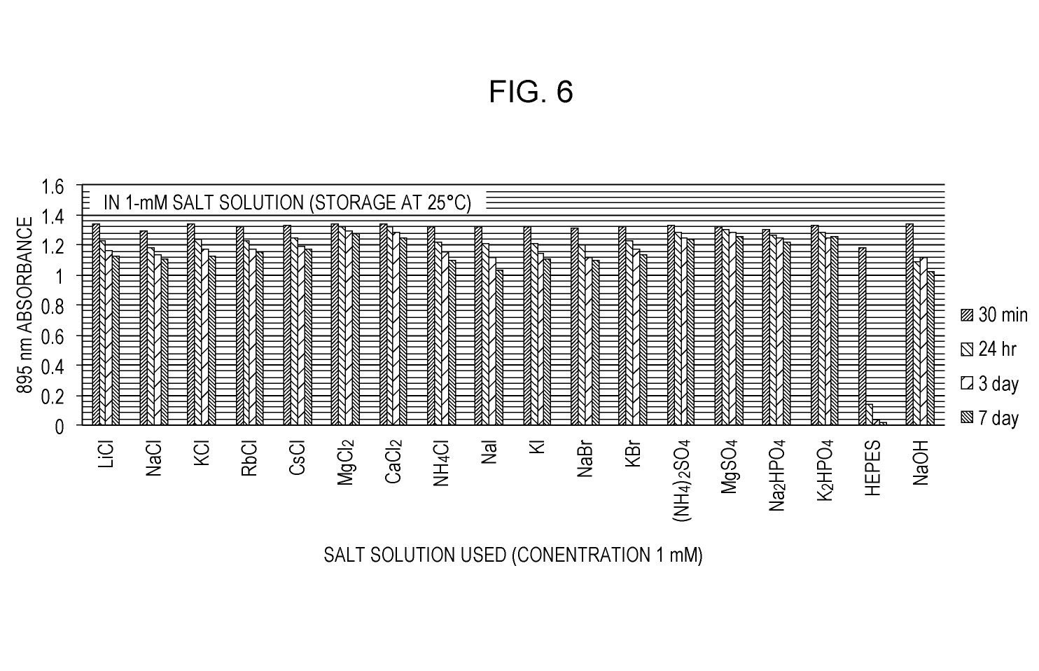

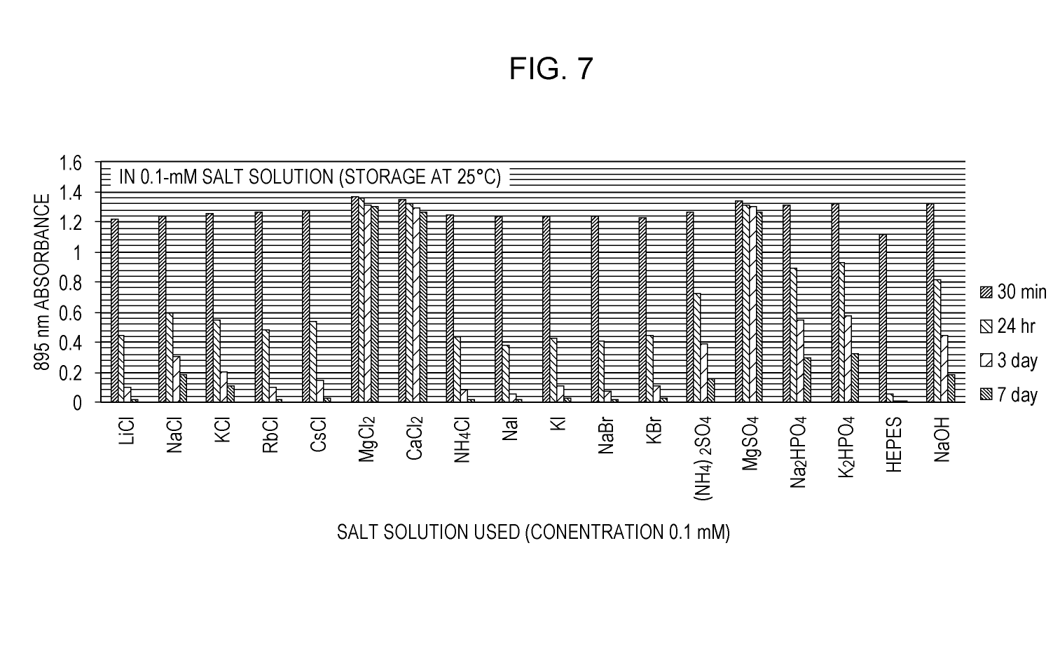

Specific examples of the composition used for contrast imaging and containing J-ICG will be described in Examples; however, reagents and reaction conditions employed in Examples can be changed, and such a change should be within the scope of the present invention. Thus, the following description in Examples is for further understanding of the present invention and does not limit the scope of the present invention at all. In FIGS. 1 to 8, the term “895 nm ABSORBANCE” herein refers to absorbance for light having a wavelength of 895 nm.

### Example 1

### Preparation of Aqueous J-ICG Solution

To 316 mg of indocyanine green (standard product, manufactured by Pharmaceutical and Medical Device Regulatory Science Society of Japan), 51 ml of water produced through deionization (hereinafter simply referred to as “water”) was added, and the solution was subjected to ultrasonic irradiation at room temperature for 10 minutes (three-cycle ultrasonic cleaner VS-100III manufactured by AS ONE Corporation). The ultrasonic irradiation was in a cycle of 60 seconds at 28 kHz, 60 seconds at 45 kHz, and 3 seconds at 100 kHz; and this cycle was repeated for 10 minutes in total to prepare an 8-mM aqueous ICG solution. The aqueous ICG solution was heated at 70° C. for 6 hours, retained at room temperature for 18 hours, and then heated again at 70° C. for 6 hours. The resulting solution was subsequently stored at 25° C. for 6 days to prepare an aqueous J-ICG solution.

### Preparation of Storage Stabilizer

A storage stabilizer used for enhancing the storage stability of the aqueous J-ICG solution was prepared. The following reagents were dissolved in water to prepare 1-M solutions, and then the pH thereof was adjusted with sodium hydroxide and hydrochloric acid: MES (2-morpholinoethanesulfonic acid, monohydrate, manufactured by DOJINDO LABORATORIES), HEPES (2-[4-(2-hydroxyethyl)-1-piperazinyl]ethanesulfonic acid, manufactured by DOJINDO LABORATORIES), PIPES (piperazine-1,4-bis(2-ethanesulfonic acid, manufactured by Sigma-Aldrich Co., LLC.), MOPS (3-morpholinopropanesulfonic acid, manufactured by DOJINDO LABORATORIES), TES (N-tris(hydroxymethyl)methyl-2-aminoethanesulfonic acid, manufactured by DOJINDO LABORATORIES), and CAPS (N-cyclohexyl-3-aminopropanesulfonic acid, manufactured by Sigma-Aldrich Co., LLC.). In particular, the pH of MES, HEPES, PIPES, MOPS, and TES was adjusted to be 7.0; and the pH of CAPS was adjusted to be 10.0. These solutions obtained by adjusting the pH were diluted with water into 10-mM solutions. These prepared reagents are referred to as “Good buffer solutions”.

Sodium dihydrogenphosphate-dihydrate (manufactured by KISHIDA CHEMICAL Co., Ltd.) and disodium hydrogenphosphate-dodecahydrate (manufactured by Wako Pure Chemical Industries, Ltd.) were separately dissolved in water to prepare 1-M aqueous solutions. These two solutions were mixed with each other at different mixing ratios to prepare 1-M phosphate buffer solutions having pH of 3.0, 7.0, or 12.1. These 1-M phosphate buffer solutions were distilled with water into 10-mM aqueous solutions. The prepared reagents are referred to as “phosphate buffer solutions”.

Citric acid monohydrate (manufactured by Wako Pure Chemical Industries, Ltd.) and trisodium citrate dihydrate (manufactured by NACALAI TESQUE, INC.) were separately dissolved in water to prepare 1-M solutions. These two solutions were mixed with each other such that the pH was 3.0, thereby preparing a 1-M citrate buffer solution. The 1-M citrate buffer solution was distilled with water into a 10-mM aqueous solution. The prepared reagent is referred to as “citrate buffer solution”.

Sodium bicarbonate (manufactured by NACALAI TESQUE, INC.) and sodium carbonate (manufactured by NACALAI TESQUE, INC.) were separately dissolved in water to prepare 1-M solutions. These two solutions were mixed with each other such that the pH was 10.0, thereby preparing a 1-M carbonate buffer solution. The 1-M carbonate buffer solution was distilled with water into a 10-mM aqueous solution. The prepared reagent is referred to as “carbonate buffer solution”.

Sodium chloride (manufactured by KISHIDA CHEMICAL Co., Ltd.) was dissolved in water to prepare a 150-mM aqueous sodium chloride solution. The prepared reagent is referred to as “saline”.

The prepared aqueous J-ICG solution was diluted 1/1000 separately with the storage stabilizers and water to prepare compositions used for contrast imaging. The prepared compositions used for contrast imaging were separately stored for a certain time under different temperature conditions of 4° C., 25° C., and 37° C.; and then absorbance for light having a wavelength of 895 nm (hereinafter referred to as OD895) was measured. In the measurement of absorbance, the aqueous J-ICG solutions diluted with the reagents were dispensed to a plate having 96 holes at 200 μL/well, and the measurement was carried out with Varioskan (manufactured by THERMO ELECTRON Co., Ltd.).

FIG. 1 shows results of the measurement. In the compositions stored at 4° C., the OD895 of the composition stored in water decreased over time, and the OD895 completely disappeared in a month. On the other hand, in all of the compositions stored in the buffer solutions or saline, the OD895 did not decrease even in a month, which showed that the J-ICG was able to be stably stored. Even when the types of buffer solutions were different from each other or even when the pH levels were different from each other within the range of pH 3.5 to 11, the J-ICG was able to be stably stored. Such solutions which enabled the stable storage of the J-ICG contained sodium ions, which showed that the sodium ions contributed to the stabilization of the J-ICG.

On the basis of the storage at higher temperatures (25° C. and 37° C.), the degrees of the solution-stabilizing effect in which the J-ICG was able to be stably stored were compared. Table 2 (storage at 25° C.) and Table 3 (storage at 37° C.) show results of the measurement. In the storage at higher temperatures, the OD895 of the compound stored in water reached approximately zero after 24 hours. The OD895 of the compounds stored in the buffer solutions or saline of which the pH was in the range of 5.1 to 10 was relatively stable even in a month, which showed that the J-ICG was able to be relatively stably stored. In the citrate buffer solution having a pH of 3.5, the phosphate buffer solution having a pH of 3.6, and the phosphate buffer solution having a pH of 11, however, a decrease in OD895 was relatively large, which showed that high-temperature storage at strong acidity or basicity reduced the stability of the J-ICG.

### Example 2

### Concentration of Sodium Chloride Necessary for Stabilization of J-ICG

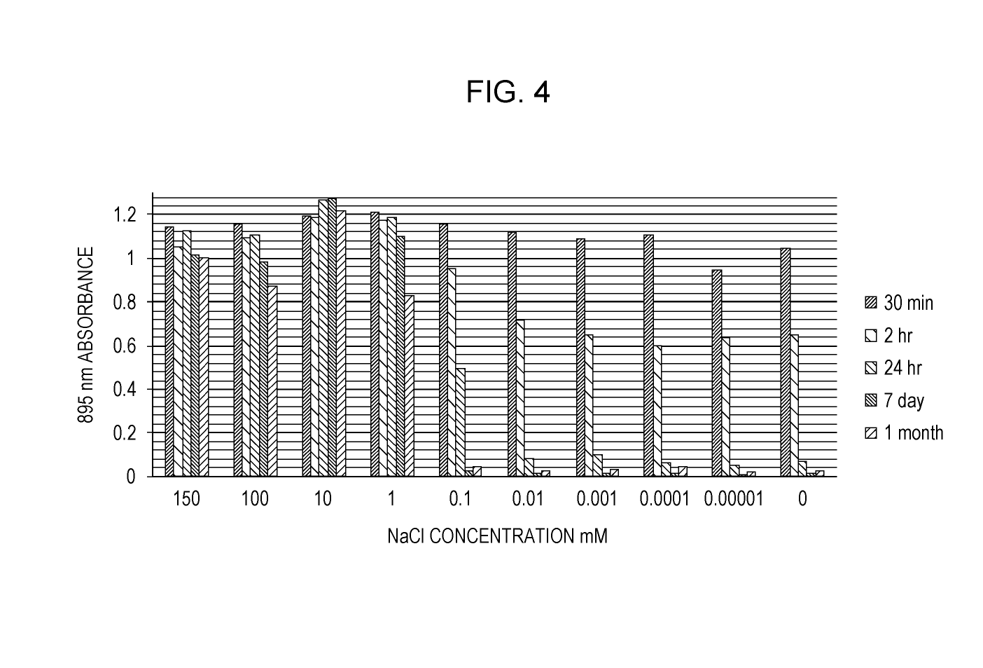

Example 1 showed that a sodium ion was effective for stabilization of the J-ICG. The effective range of sodium ion concentration for stabilization of the J-ICG was analyzed, and FIG. 4 shows results of the analysis.

The process in Example 1 was employed, and aqueous NaCl solutions having different concentrations were used in place of saline (NaCl, 150 mM) to measure OD895 over time under a storage condition of 25° C.

An aqueous J-ICG solution in which the molar concentration of ICG was 8 μM was able to be stably stored over a month at an NaCl concentration of not less than 1 mM. It was particularly effective to use NaCl at a molar concentration at least 125 times the molar concentration of ICG, which showed that use of NaCl at a molar concentration at least approximately 100 times the molar concentration of ICG especially enabled stable storage of the J-ICG.

### Example 3

### Stabilization Effect of Use of Various Cations on J-ICG

A sodium ion is an alkali metal ion, and a test was carried out to confirm whether other alkali metal ions had a similar effect or not. In addition, the stabilization effects of alkaline earth metal ions and ammonium ion were similarly investigated.

The process in Example 1 was employed; aqueous solutions of a variety of alkali metal salts, alkaline earth metal salts, and ammonium salts were used in place of saline; an aqueous HEPES solution which was free from sodium hydroxide was used for comparison; and then OD895 over time under a storage condition of 25° C. was measured. The stabilization effects on the J-ICG were compared at concentrations of the salt solutions of 10, 1, and 0.1 mM.

The alkali metal salts used were lithium chloride (manufactured by KANTO CHEMICAL CO., INC.), sodium chloride, potassium chloride, rubidium chloride, cesium chloride, sodium iodide, potassium iodide, sodium bromide, potassium bromide, disodium hydrogen-phosphate, dipotassium hydrogen-phosphate, and sodium hydroxide (each manufactured by KISHIDA CHEMICAL Co., Ltd.). These salts were dissolved in water to prepare aqueous solutions having concentrations of 10, 1, or 0.1 mM. After the J-ICG was added, the pH of the aqueous solutions of the chloride salts, iodide salts, and bromide salts (10 mM) was in the range of 5.3 to 5.6; the pH of the aqueous solutions of phosphates (10 mM) was in the range of 8.8 to 9.8; and the pH of the aqueous solution of sodium hydroxide (10 mM) was 11.6.

The alkaline earth metal salts used were magnesium chloride (manufactured by KISHIDA CHEMICAL Co., Ltd.), calcium chloride (manufactured by NACALAI TESQUE, INC.), and magnesium sulfate (manufactured by Wako Pure Chemical Industries, Ltd.). These were dissolved in water to prepare aqueous solutions having concentrations of 10, 1, or 0.1 mM. After the J-ICG was added, the pH of these solutions was in the range of 5.4 to 5.7.

Ammonium salts used were ammonium chloride (manufactured by KISHIDA CHEMICAL Co., Ltd.) and ammonium sulfate (manufactured by Wako Pure Chemical Industries, Ltd.). These were dissolved in water to prepare aqueous solutions having concentrations of 10, 1, or 0.1 mM. After the J-ICG was added, the pH of these solutions was in the range of 5.3 to 5.4.

HEPES (manufactured by DOJINDO LABORATORIES) was dissolved in water to prepare aqueous HEPES solutions having concentrations of 10, 1, or 0.1 mM. After the J-ICG was added, the pH of these solutions was 5.3.

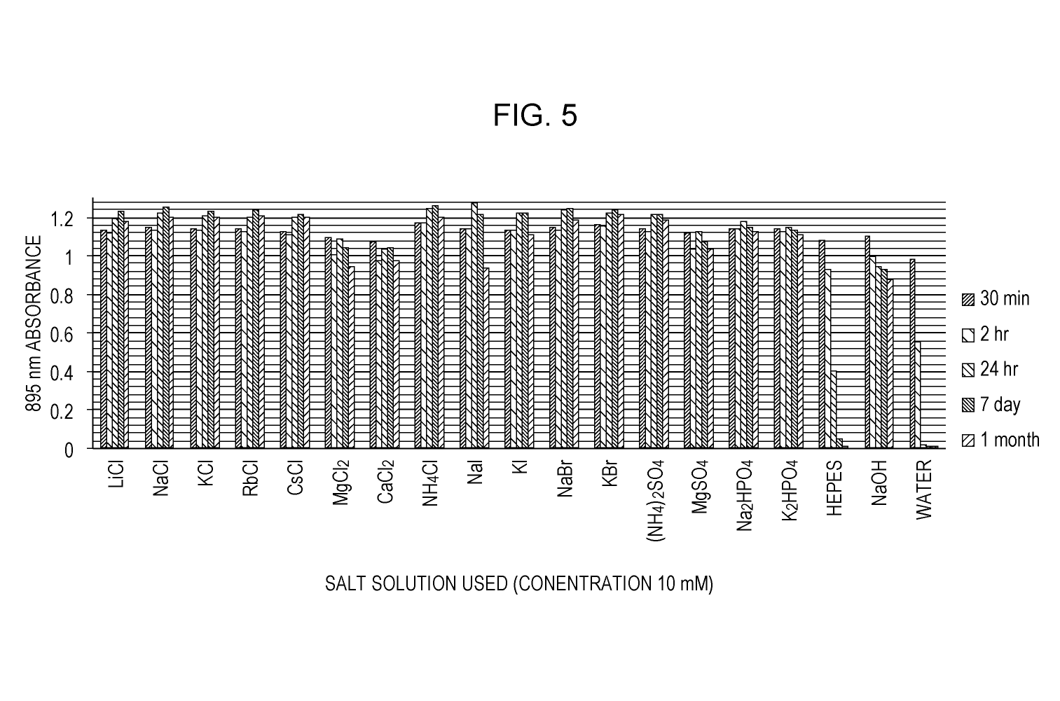

FIG. 5 shows stabilization effects of the 10-mM salts in the aqueous solutions on the J-ICG over time.

As in Examples 1 and 2, OD895 substantially disappeared after 24 hours in the storage in water. In all of the aqueous alkali metal salt solutions, a large decrease in OD895 had not been observed at 25° C. over a month, which showed that the J-ICG was able to be stably stored. In addition, the aqueous alkaline earth metal salt solutions and the aqueous ammonium salt solutions similarly had a high stabilization effect on the J-ICG as in alkali metal salts.

In the aqueous HEPES solution which was free from sodium ions, however, the stabilization effect on the J-ICG, which the HEPES buffer solution had in Example 1, was not observed. This result showed that the stabilization effect of the HEPES buffer solution on the J-ICG in Example 1 was owing to sodium ions added for the pH adjustment. In other words, even in the case where a buffer agent such as HEPES was added to an aqueous alkali metal salt solution, the stabilization effect on the J-ICG was maintained as in the stabilization effect of the HEPES buffer solution on the J-ICG ICG in Example 1.

The above-mentioned experiment was similarly carried out except that the concentration of the aqueous salt solutions was changed. In Example 2, in the case where the molar concentration of sodium chloride is 125 times the molar concentration of ICG, namely in a 1-mM aqueous salt solution, the stabilization effect was produced. Table 6 shows results of an experiment involving use of 1-mM aqueous solutions in which sodium chloride produced the stabilization effect. Table 7 shows results of an experiment involving use of 0.1-mM aqueous solutions which had a relatively small stabilization effect.

At the concentration of 1 mM, OD895 did not greatly decrease even in the case of using alkali metal ions other than a sodium ion, and the stabilization effect on the J-ICG was able to be observed as in use of a sodium ion. At the concentration of 0.1 mM, use of alkali metal ions other than a sodium ion also produced the effect on storage stability as in use of a sodium ion; however, OD895 decreased as compared to the concentration of 1 mM and the concentration of 10 mM.

Use of any alkali metal ion was able to produce the equivalent stabilization effect. Furthermore, even use of an ammonium ion produced the effect equivalent to the effect produced by alkali metal ions.

Alkaline earth metal ions also had the stabilization effect as in alkali metal ions; however, OD895 did not decrease even at a concentration of 0.1 mM, which showed that the stabilization effect was produced even at lower concentration.

### Example 4

### Concentration of Alkaline Earth Metal Salt Necessary for Stabilization of J-ICG

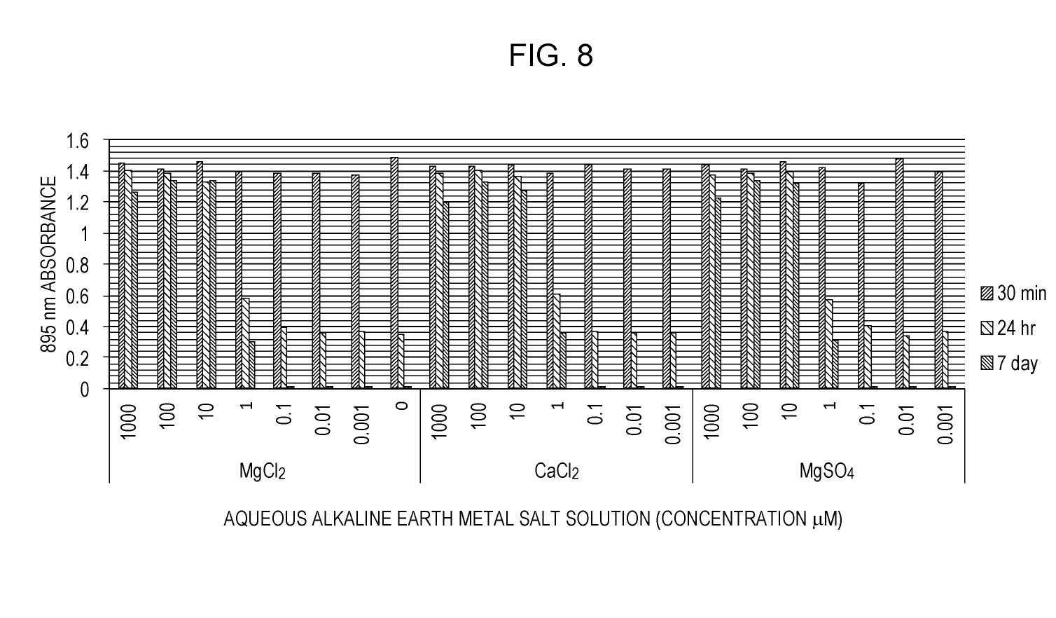

Example 3 showed that alkaline earth metal ions produced the stabilization effect on the J-ICG at a lower concentration as compared to alkali metal ions. The effective range of alkaline earth metal ion concentration for the stabilization of the J-ICG was analyzed, and FIG. 8 shows results of the analysis.

The process in Example 1 was employed, and aqueous alkaline earth metal salt solutions having different concentrations were used instead of saline (NaCl, 150 mM) to measure OD895 over time at a storage condition of 25° C. The alkaline earth metal salts used were magnesium chloride, calcium chloride, and magnesium sulfate.

In an aqueous J-ICG solution in which the molar concentration of the ICG was 8 μM, both use of a magnesium ion and use of a calcium ion produced the stabilization effect at a concentration of not less than 10 μM. It was particularly effective to use an alkaline earth metal ion at a molar concentration at least 1.25 times the molar concentration of ICG, which showed that use of an alkaline earth metal ion at a molar concentration at least approximately 1 time the molar concentration of ICG especially enabled stable storage of the J-ICG.

The composition used for contrast imaging according to the present invention contained at least one selected from the group consisting of alkali metal ions, alkaline earth metal ions, and an ammonium ion; thus, dissociation of J-ICG in water was able to be reduced, and storage stability was therefore enhanced.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-171646 filed Aug. 21, 2013, which is hereby incorporated by reference herein in its entirety.

What is claimed is:

1. A composition used for contrast imaging, the composition comprising:
an aqueous solution containing a J-aggregate of indocyanine green; and
at least one storage stabilizer selected from the group consisting of alkali metal ions, alkaline earth metal ions, and an ammonium ion.

2. The composition according to claim 1, wherein the storage stabilizer is at least one selected from the group consisting of a sodium ion, a potassium ion, a magnesium ion, a calcium ion, and an ammonium ion.

3. The composition according to claim 1, wherein the molar concentration of the storage stabilizer is at least 1 time the molar concentration of the indocyanine green.

4. The composition according to claim 1, wherein the molar concentration of the storage stabilizer is at least 1.25 times the molar concentration of the indocyanine green.

5. The composition according to claim 1, wherein the molar concentration of the storage stabilizer is at least 100 times the molar concentration of the indocyanine green.

6. The composition according to claim 1, wherein the J-aggregate of indocyanine green is in the form of a particle, and the particle size is in the range of 1 nm to 10 μm.

7. The composition according to claim 1, wherein the J-aggregate of indocyanine green is in the form of a particle, and the particle size is not more than 100 nm.

8. A composition used for photoacoustic imaging, the composition comprising:
the composition used for contrast imaging according to claim 1; and
a dispersion medium.
